(19) United States
(12) Patent Application Publication
(10) Pub. No.: US 2024/0285150 A1
(43) Pub. Date: Aug. 29, 2024

(54) SYSTEMS AND METHODS FOR ENDOSCOPE PROXIMAL END DESIGN

(71) Applicant: Noah Medical Corporation, San Carlos, CA (US)

(72) Inventors: Ka Chun WONG, South San Francisco, CA (US); Maziyar KESHTGAR, San Jose, CA (US)

(21) Appl. No.: 18/652,456

(22) Filed: May 16, 2024

Related U.S. Application Data
(63) Continuation of application No. PCT/US2022/051086, filed on Nov. 28, 2022.
(60) Provisional application No. 63/284,825, filed on Dec. 1, 2021.

(51) Int. Cl. A61B 1/00; A61B 1/005; A61B 34/00; A61B 34/30; A61M 25/01

(52) U.S. Cl. CPC A61B 1/00105; A61B 1/00103; A61B 1/0057; A61B 34/71; A61B 2034/301; A61B 2034/715; A61M 25/0147

(57) ABSTRACT

An articulating flexible endoscope is provided. The endoscope comprises: a proximal portion comprising a driving mechanism for applying a force to a pull wire, the pull wire for articulating a bending section of the articulating flexible endoscope upon the force; and an anchoring mechanism coupled to the driving mechanism. A terminal end of the pull wire is configured to wind around the anchoring component with a pre-determined number of windings thereby reducing a holding tension at the terminal end of the pull wire.

### REFERENCE

This application is a continuation of International Application No. PCT/US2022/051086, filed Nov. 28, 2022, which claims priority to U.S. Provisional Patent Application No. 63/284,825, filed on Dec. 1, 2021, which is entirely incorporated herein by reference.

### BACKGROUND OF THE INVENTION

Endoscopy procedures use an endoscope to examine the interior of a hollow organ or cavity of the body. Unlike many other medical imaging techniques, endoscopes are inserted into the organ directly. Flexible endoscope that can deliver instinctive steering and control is useful in diagnosing and treating diseases that are accessible through any natural orifice in the body. Depending on the clinical indication, the endoscope may be designated as bronchoscope, ureteroscope, colonoscope, gastroscope, ENT scope, and various others. For example, flexible endoscopy has been used to inspect and treat disorders of the gastrointestinal (GI) tract without the need for creating an opening on the patient's body. The endoscope is introduced via the mouth or anus into the upper or lower GI tracts respectively. A miniature camera at the distal end captures images of the GI wall that help the clinician in their diagnosis of the GI diseases. Simple surgical procedures (like polypectomy and biopsy) can be performed by introducing a flexible tool via a working channel to reach the site of interest at the distal end.

Endoscopes may be articulated for controlling a direction of the endoscope. In some cases, articulation of the endoscope may be controlled by applying force to the distal end of the endoscope via one or multiple pull wires. For example, pulling one wire at a time may change the orientation of the distal tip to pitch up, down, left, right or any direction needed. In some cases, the pull wires may be anchored at the distal tip of the endoscope, running through the bending section, and entering the handle (proximal end) where they are coupled to a driving component (e.g., pulley, capstans). However, conventional methods of terminating or anchoring pull wires to pulleys or capstans (e.g., crimping) may have a holding strength less than tensile strength of the pull wire itself in medical instruments. This may result in wire breaks at capstan interface since wire tension is highest at the capstan.

### SUMMARY OF THE INVENTION

Recognized herein is a need for an improved pull wire termination or anchoring design. The present disclosure provides methods and apparatuses for a pull wire anchoring mechanism with improved reliability without introducing additional cost. The terminating mechanism may allow for utilization of the full tensile strength of the pull wire or reduction of the holding tension. Additionally, the present disclosure provides the proximal design that can be incorporated into a low-cost, single-use articulatable endoscope for diagnosis and treatment in various applications such as bronchoscopy, urology, gynecology, arthroscopy, orthopedics, ENT, gastro-intestine endoscopy, neurosurgery, and various others. It should be noted that the provided endoscope systems can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

It should be noted that the provided pull wire anchoring mechanism and various components of the device can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

In an aspect of the present disclosure, an articulating flexible endoscope is provided. The articulating flexible endoscope comprises: a proximal portion comprising a driving mechanism for applying a force to a pull wire; the pull wire for articulating a bending section of the articulating flexible endoscope upon the force; and an anchoring mechanism coupled to the driving mechanism, wherein a proximal end of the pull wire is configured to wind around the anchoring component with a pre-determined number of windings thereby reducing a holding tension at the proximal end of the pull wire.

In some embodiments, the driving mechanism comprises a capstan. For instance, the capstan comprises a cutout to receive the anchoring mechanism. In some cases, the anchoring mechanism is freely rotatable within the cutout. For example, the anchoring mechanism is rotated to adjust the number of windings. In some embodiments, the anchoring mechanism is coupled to the driving mechanism via a screw. In some cases, the anchoring mechanism and the cutout have a non-circular shape such that the anchoring mechanism is not rotatable within the cutout.

In some embodiments, the pre-determined number of windings are determined based at least in part on a load requirement and a coefficient of friction between the pull wire and the anchoring mechanism. In some embodiments, the articulating flexible endoscope further comprises a distal tip portion embedded with an imaging device, a position sensor and an illumination device. In some embodiments, the entire articulating flexible endoscope is disposable or single-use.

In another aspect, a method is provided for coupling a pull wire to an articulating flexible endoscope. The method comprises: providing an anchoring mechanism; wrapping a proximal end of the pull wire to the anchoring mechanism with a pre-determined number of windings thereby reducing a holding tension at the proximal end of the pull wire; and assembling the anchoring mechanism to a driving mechanism located at a proximal portion of the articulating flexible endoscope.

In some embodiments, the driving mechanism is configured for applying a force to a pull wire. In some cases, a bending section of the articulating flexible endoscope is articulated by the pull wire upon the force.

In some embodiments, the driving mechanism comprises a capstan. In some cases, the capstan comprises a cutout to receive the anchoring mechanism. In some cases, the anchoring mechanism is freely rotatable within the cutout. For instance, the method may comprise further adjusting the number of windings by rotating the anchoring mechanism.

In some embodiments, the method further comprises determining the pre-determined number of windings based at least in part on a load requirement and a coefficient of friction between the pull wire and the anchoring mechanism. In some embodiments, the articulating flexible endoscope comprises a distal tip portion embedded with an imaging device, a position sensor and an illumination device. In some cases, the entire articulating flexible endoscope is disposable or single-use.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example of a flexible endoscope, in accordance with some embodiments of the present disclosure.

FIG. 2 shows an example of an endoscope with an articulation force transmission mechanism and pull wires, in accordance with some embodiments of the invention.

FIG. 3 shows an example of a proximal portion of an endoscope where one or more pull wires are terminated.

FIG. 4 shows an example of an anchoring mechanism, in accordance with some embodiments of the present disclosure.

FIG. 5 shows an example of fixing the terminal end of a pull wire to an insert component.

FIG. 6 shows an example of coupling an insert component to a capstan.

FIGS. 7-9 show examples of a proximal portion of the endoscope comprising a plurality of capstans and a plurality of corresponding anchoring mechanism.

FIG. 10 shows an example of a robotic bronchoscope comprising a handle portion and a flexible elongate member.

FIG. 11 shows an example of an instrument driving mechanism (IDM) providing mechanical interface to the handle portion of the robotic bronchoscope.

FIG. 12 shows an example of a distal tip of an endoscope.

FIG. 13 shows an example distal portion of a catheter with integrated imaging device and an illumination device.

FIG. 14 shows an example of a disposable endoscope removably coupled to an IDM.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

While exemplary embodiments will be primarily directed at a device or system for bronchoscopy, one of skill in the art will appreciate that this is not intended to be limiting, and the devices described herein may be used for other therapeutic or diagnostic procedures and in various anatomical regions of a patient's body. The provided device or system can be utilized in urology, gynecology, rhinology, otology, laryngoscopy, gastroenterology with the endoscopes, combined devices including endoscope and instruments, endoscopes with localization functions, one of skill in the art will appreciate that this is not intended to be limiting, and the devices described herein may be used for other therapeutic or diagnostic procedures and in other anatomical regions of a patient's body, such as such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat, and various others, in the forms of: NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, Orthopedic Endoscopes, and others, in combination with various tools or instruments.

The systems and apparatuses herein can be combined in one or more of many ways to provide improved diagnosis and therapy to a patient. Systems and apparatuses provided herein can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms distal and proximal may generally refer to locations referenced from the apparatus, and can be opposite of anatomical references. For example, a distal location of a primary shaft or catheter may correspond to a proximal location of an elongate member of the patient, and a proximal location of the primary sheath or catheter may correspond to a distal location of the elongate member of the patient.

Endoscopes may be articulated for controlling a direction of the endoscope. In some cases, articulation of the endoscope may be controlled by applying force to the distal end of the endoscope via one or multiple pull wires. For example, pulling one wire at a time may change the orientation of the distal tip to pitch up, down, left, right or any direction needed. In some cases, a distal end of the pull wire may be anchored at the distal tip of the endoscope, running through the bending section, and entering the handle (proximal end) where a proximal end of the pull wire may be coupled to a driving component (e.g., pulley, capstans). Conventional method (e.g., crimping) of coupling the terminal end of the pull wire to the driving component (e.g., capstan) may result in wire breaks at capstan interface since wire tension is highest at the capstan. Wire breaks at the capstan interface is a common failure mode as wire tension is highest at the capstan and decreases as towards the distal end of the instrument due to friction stack up in the system.

The present disclosure provides a mechanism for anchoring the pull wire(s) to the driving mechanism/component at the proximal end of an endoscope. In some embodiments, the anchoring mechanism may comprise an insert component where a pull wire may be coupled to the insert by wrapping around the insert. The anchoring mechanism may beneficially reduce the holding tension on the terminated side thereby improving the reliability of the system. In some cases, the insert component may be coupled to the capstan with a feature to prevent it from moving relative to the capstan. The mechanism may also allow for a simplified assembly process without introducing additional cost.

### Flexible Endoscope

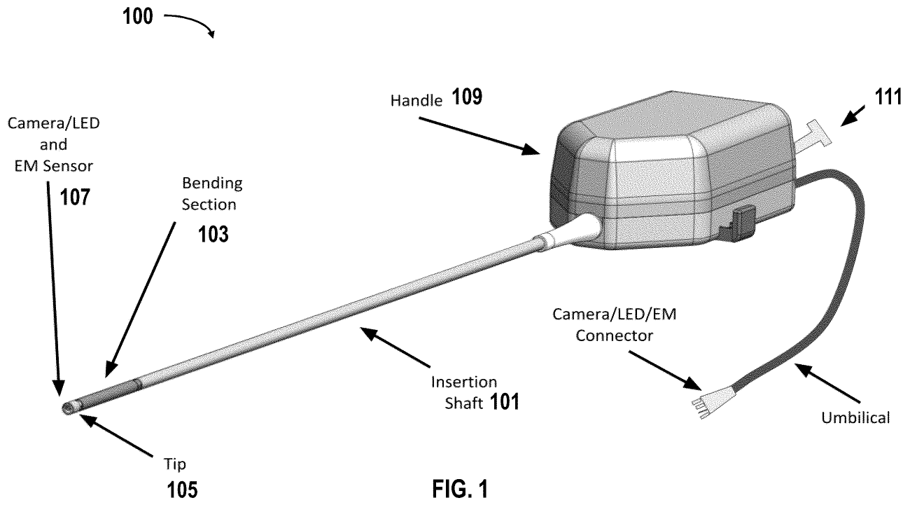

In some embodiments, the anchoring mechanism herein may be utilized for improving reliability at a proximal end of a flexible endoscope. The provided anchoring mechanism may be utilized by any devices or apparatuses where a pull wire is employed. In an aspect of the invention, a flexible endoscope with improved performance (e.g., improved reliability) at reduced cost is provided. FIG. 1 illustrates an example of a flexible endoscope 100, in accordance with some embodiments of the present disclosure. As shown in FIG. 1, the flexible endoscope 100 may comprise a handle/proximal portion 109 and a flexible elongate member to be inserted inside of a subject. In some embodiments, the flexible elongate member may comprise a shaft (e.g., insertion shaft 101), steerable tip (e.g., tip 105) and a steerable section (bending section 103). The endoscope 100 may also be referred to as steerable catheter assembly as described elsewhere herein. In some cases, the endoscope 100 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiment, the endoscope may contain varying levels of stiffness along the shaft, as to improve functional operation.

The endoscope or steerable catheter assembly 100 may comprise a handle portion 109 that may include one or more components configured to process image data, provide power, or establish communication with other external devices. For instance, the handle portion may include a circuitry and communication elements that enables electrical communication between the steerable catheter assembly 100 and an instrument driving mechanism (IDM), and any other external system or devices. In another example, the handle portion 109 may comprise circuitry elements such as power sources for powering the electronics (e.g., camera, electromagnetic sensor and LED lights) of the endoscope.

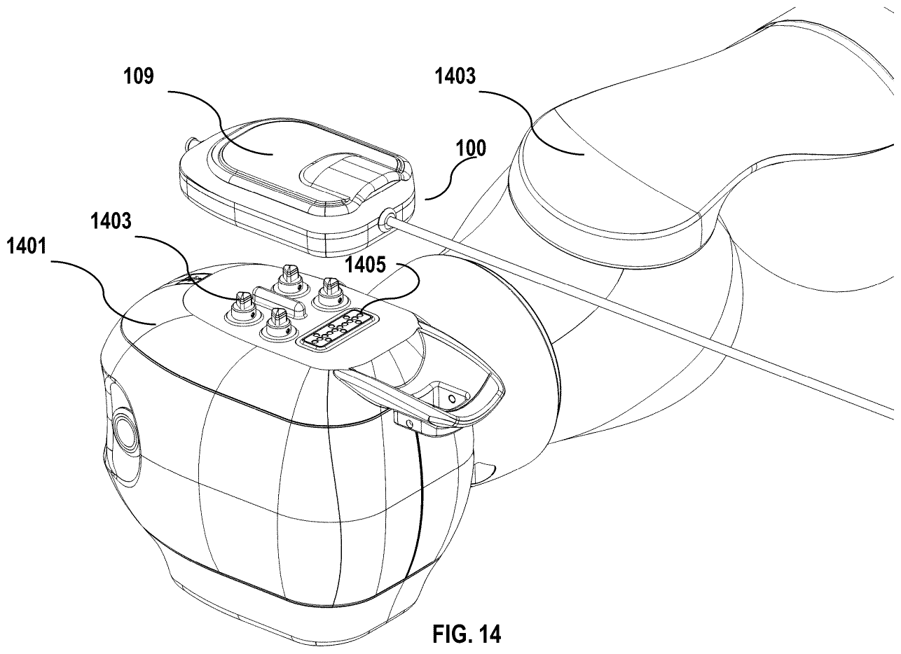

FIG. 14 shows an example of a disposable endoscope 100 removably coupled to an IDM 1401. The one or more components located at the handle 109 may be optimized such that expensive and complicated components may be allocated to the robotic support system 1403, a hand-held controller or an instrument driving mechanism 1401 thereby reducing the cost and simplifying the design the disposable endoscope. The handle portion or proximal portion 109 may provide an electrical interface and mechanical interface to allow for electrical communication and mechanical communication with the instrument driving mechanism 1401. The instrument driving mechanism 1401 may comprise a set of motors that are actuated to rotationally drive a set of pull wires of the catheter. The handle portion 109 of the catheter assembly may be mounted onto the instrument drive mechanism 1401 so that its pulley/capstans assemblies are driven by the set of motors. For example, the handle pulley may interact with an output shaft 1403 from the IDM supported by the robotic system The number of pulleys may vary based on the pull wire configurations. In some cases, one, two, three, four, or more pull wires may be utilized for articulating the flexible endoscope or catheter.

The handle portion may be designed allowing the robotic endoscope (e.g., bronchoscope) to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

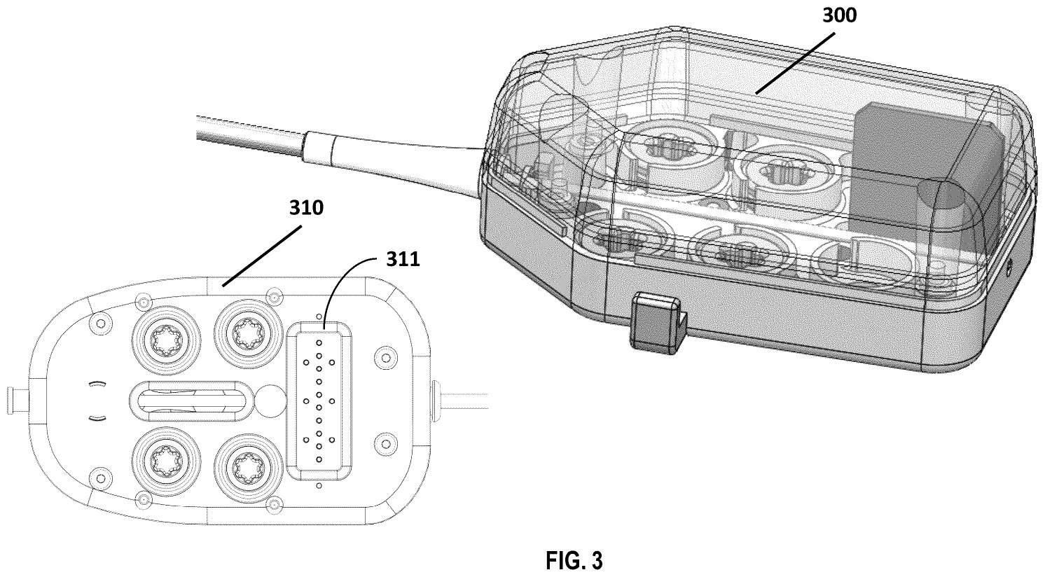

As shown in the bottom view 310 of the handle in FIG. 3, the electrical interface (e.g., printed circuit board) 311 may allow image/video data and/or sensor data to be received by the communication module of the instrument driving mechanism and may be transmitted to other external devices/systems. In some cases, the electrical interface 311 may establish electrical communication without cables or wires. For example, the interface may comprise pins soldered onto an electronics board such as a printed circuit board (PCB). For instance, receptacle connector (e.g., the female connector 1405 in FIG. 14) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

Referring back to FIG. 1, in some cases, the handle portion 109 may comprise one or more mechanical control modules such as lure 111 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 100. The mechanical interface may allow the steerable catheter assembly 100 to be releasably coupled to the instrument driving mechanism. For instance, the handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool. Details about the instrument driving mechanism are described later herein.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor) 107 is located at the tip of the catheter or endoscope shaft 105. For example, line of sight of the camera may be controlled by controlling the articulation of the bending section 103. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with aid of an optimal component.

The distal tip 105 may be a rigid component that allow for positioning sensors such as electromagnetic (EM) sensors, imaging devices (e.g., camera) and other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5D or 6D space. This may provide a visual guide to an operator when driving the bronchoscope towards the target site. Details about the tip design and the plurality of components embedded at the tip are described later herein.

The endoscope may have a unique design in the shaft component. In some cases, the insertion shaft of the endoscope may consist of a single tube that incorporates a series of cuts (e.g., reliefs, slits, etc.) along its length to allow for improved flexibility as well as a desirable stiffness.

The bending section 103 may be designed to allow for bending in two or more degrees of freedom (e.g., articulation). A greater bending degree such as 180 and 270 degrees (or other articulation parameters for clinical indications) can be achieved by the unique structure of the bending section. In some cases, the bending section may be fabricated separately as a modular component and assembled to the insertion shaft. In some cases, the bending section may further incorporate minimalist features thereby reducing cost and increasing reliability. For example, the bending section may incorporate a cut pattern that beneficially allows for a greater degree of tube deflection to achieve a desired tip displacement relative to the insertion shaft.

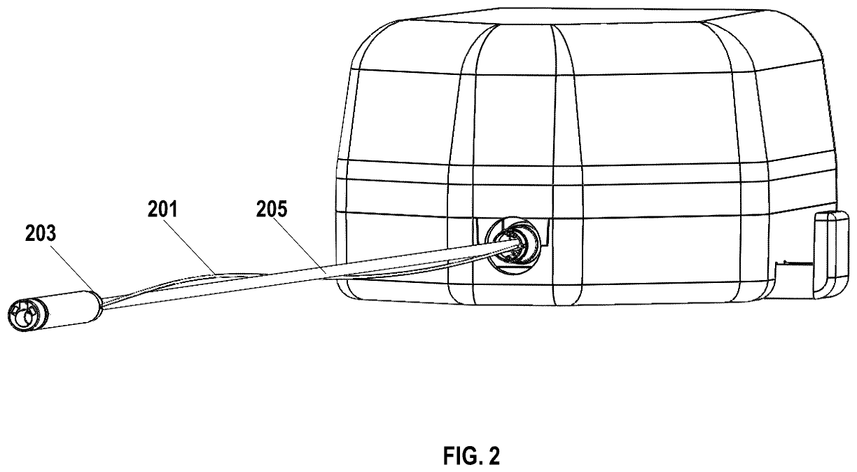

In some embodiments, the bending section or the endoscope may comprise an articulation force transmission mechanics to ensure that the endoscope is stable and delivers instinctive bending section responsiveness. A pull wire may be positioned inside of a load transmission tube (e.g., hypotube). FIG. 2 shows an example of an endoscope with an articulation force transmission mechanism 201 with pull wires passing through, in accordance with some embodiments of the invention. The articulation force transmission mechanism 201 may include a plurality of load transmission tubes that are located inside the bore of the insertion shaft/tube (not shown). In some cases, at least one, two, three, four, five or more load transmission tubes may be included to reduce the axial compression/extension (strain) of the insertion tube during articulation of the bending section. The load transmission tubes may transmit at least a portion of the articulation load applied to the bending section and/or the shaft back to the handle (e.g., via actuator or motors that drive one or more articulating pull wires). In some embodiments, the one or more load transmission tubes may enclose one or more pull wires.

The shaft portion may comprise one or more load transmission tubes for accommodating the one or more pull wires passing through the load transmission tubes. The load transmission tube and pull wire may be terminated at a distal shaft region 203 and proximal shaft region. As described elsewhere herein, the load transmission tube may be located within the lumen of the insertion shaft (not shown) and external to the working channel 205.

The transmission tubes counteract the articulation loads allowing for an improved stability of the insertion shaft. The plurality of load transmission tubes 201 may reside within the lumen of the shaft tube (i.e., tube bore) and be configured to transfer articulation reaction forces from the bending section to the handle portion. The load transmission tubes are configured to transfer the bending section articulation reaction forces back to the handle portion thereby reducing the articulation forces that would have been applied to the insertion shaft tube. Such design may beneficially prevent these articulation forces from being resolved through the insertion shaft tube thus providing a stable shaft. The transmission modality described herein may ensure that the insertion shaft tube experiences minimal axial compressive or extension forces, thereby remaining stable during the articulation of the bending section.

In preferred embodiments of the load transmission mechanism, the plurality of load transmission tubes 201 may be longer than the length of the insertion shaft tube or working channel 203. The length of the plurality of load transmission tubes 201 may be determined such that when the load transmission tubes are under axial compression, they are still longer than the length of the insertion shaft tube thereby preventing loads from transferring through the insertion shaft tube. For example, the length of the load transmission tubes may be at least 0.01%, 0.1%, 0.2%, 0.3%, 1%, 5%, 10% longer than the length of the insertion shaft. The length of the load transmission tubes may be determined based at least in part on the dimension of the inner diameter of the shaft. For example, the load transmission tubes may have a spiral configuration that provides sufficient stiffness to bear/transmit the load.

The articulation of the endoscope may be controlled by applying force to the distal end of the endoscope via one or multiple pull wires. In some embodiments, the one or more load transmission tubes may enclose one or more pull wires. FIG. 3 shows an example of a proximal portion 300 of the endoscope where the one or more pull wires are terminated. A distal end of the one or more pull wires may be attached to the distal end of the endoscope. In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle 300 where they are coupled to a driving component (e.g., pulley). As described above, the handle pulley may interact with an output shaft from the instrument driving mechanism.

In some embodiments, the one or more pull wires may be located within a lumen of the one or more load transmission tubes or running through the inside of the transmission tubes. For example, a driving mechanism (e.g., actuators, motors) may be engaged with a capstan to which the pull wire is terminated to articulate the bending section. The one or more load transmission tubes may be configured to transmit at least a portion of the articulation loads (e.g., compression) from the bending section back to the handle or motors, for example, by placing the one or more pull wires inside the one or more load transmission tubes, respectively. There may be relative motion between the pull wire and the corresponding load transmission tube during articulation. The one or more load transmission tubes may transmit at least a portion of the articulation load applied to the bending section and/or the shaft back to the handle (e.g., motors that drive one or more articulating pull wires). This may beneficially reduce at least a portion of the articulation force applied to the bending section and/or the insertion shaft thereby improving stability of the insertion shaft.

In some cases, the one or more load transmission tubes may be composed of materials such as metallic tubing or metallic wound coil pipe. The geometry and/or materials of the load transmission tubes may be selected/determined to provide desired axial and bending stiffness. For example, the material may be metallic materials such as stainless steel or nitinol, stiff polymers such as PEEK, glass or carbon filled PEEK, Ultem, Polysulfone and other suitable materials. In some cases, the one or more load transmission tubes may have an inner diameter greater than the outer diameter of the pull wire to allow for relative movement (e.g., translational and/or rotational movement) between the load transmission tubes and the pull wire. The wall thickness of the one or more load transmission tubes may be determined based on a function of the load transmission needed to transfer the articulation loads of the bending section.

In some embodiments, the proximal end or portion of one or more pull wires may be operatively coupled to various mechanisms (e.g., gears, pulleys, capstans, etc.) in the handle portion of the catheter assembly. The proximal end or terminal end of the one or more pull wires may be anchored to the driving mechanism using the anchoring method or devices provided herein.

A pull wire may be a metallic wire, cable or thread, or it may be a polymeric wire, cable or thread. A pull wire can also be made of natural or organic materials or fibers. A pull wire can be any type of suitable wire, cable or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g. SS316), metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals. Pull wires may run through the lumen of one or more load transmission tubes and/or hypotubes as described elsewhere herein.

### Pull Wire Anchoring Mechanism

In one aspect, the present disclosure provides a mechanism for anchoring the pull wire(s) at the proximal end of an endoscope. In some embodiments, the mechanism may comprise an insert component. A proximal end of a pull wire may be coupled to the insert component and wrapped around the insert component. The anchoring mechanism may beneficially reduce the holding tension on the terminated side thereby improving the reliability of the system. In some cases, the insert component may be coupled to the driving mechanism (e.g., capstan) with a feature to prevent it from moving relative to the capstan. The mechanism may also allow for a simplified assembly process without introducing additional cost.

Method and devices herein may significantly reduce the holding tension in the pull wire thereby improving the reliability of the system. The holding tension may be reduced by wrapping the terminal end of the pull wire around an insert component.

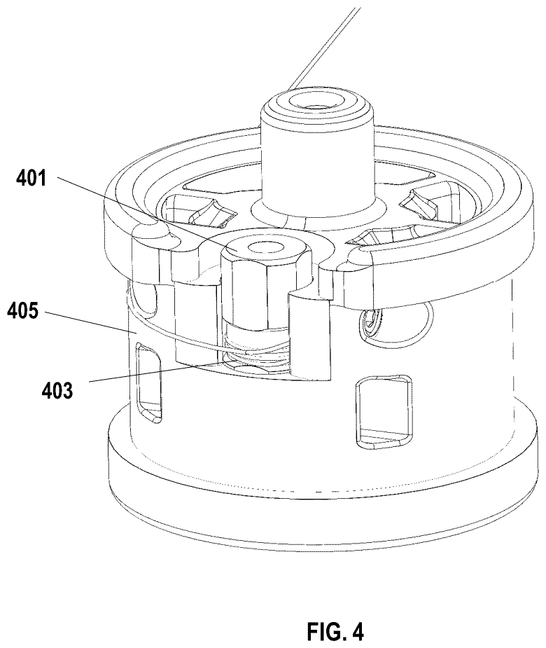

FIG. 4 shows an example of an anchoring mechanism, in accordance with some embodiments of the present disclosure. The anchoring mechanism may comprise an insert component 401. A terminal or proximal end of a pull wire 403 may wrap around the insert component 401. The insert component 401 may be coupled to a capstan 405 such as by embedding into a recessive structure or a cutout of the capstan. A terminal end of the pull wire may be fixed to the anchoring mechanism by wrapping around the insert component 401 prior to wrapping around the capstan.

As shown in FIG. 4, a holding tension in the pull wire may be reduced with aid of the friction between the insert 401 and the wraps of the pull wire 403. The holding tension in the pull wire 403 may be exponentially decreased with an increased number of wire wraps and coefficient of friction between the wire and the surface (of the insert) in contact. With sufficient number of wire wraps around (e.g., number of windings) the insert and high enough coefficient of friction, the tension required to break the anchoring to the insert within the capstan may need to exceed the tensile strength of the pull wire. This beneficially allows for utilizing the full tensile strength of the pull wire and increasing the fatigue life of the termination.

The holding tension required to support the tension in the pull wire may depend on the windings (e.g., number of wraps/windings) and the coefficient of friction. The method herein may comprise determining a minimum number of wraps and the material of the insert component to reduce the holding tension while meeting a load requirement (e.g., maximum load requirement). In some cases, the minimum number of wraps may be determined based at least in part on Euler-Eytelwein equation. Below shows an example of the Euler-Eytelwein equation for calculating the exponential decrease in holding tension of the insert (or pull wire) with an increase in number of wraps and friction:

$${T}_{l o a d} = {T}_{h o l d} {e}^{μ θ}$$

where T_{load} represents the tension in the pull wire due to the load/force applies to it, T_{hold} represents the tension necessary to hold the load without slipping, p is the coefficient of friction between the wire and the insert, and θ is the total angle, in radians, made by all the windings of the pull wire.

The provided anchoring mechanism may significantly reduce the holding tension. For example, wrapping a pull wire with PTFE coating around an insert made of steel with eight windings may reduce the holding tension by at least 45%. The holding tension can be reduced by at least 30%, 40%, 50%, 60%, 70%, 80%, 90%, or more depending on the selection of the number of windings and/or the coefficient of friction. In an example of requiring an anchoring point to withstand 50N load, if the coefficient of friction is 0.18 (e.g., a PTFE-coated pull wire is wrapped around an insert made of steel), with a number of 8 wraps/windings around the insert (i.e., 16π radians), the holding tension in the pull wire may be calculated based on the Euler-Eytelwein equation as, T_{load}=50 N, μ=0.18, and θ=16π: 50=T_{hold}(e^{(0.18)(16π)}), T_{hold}=50/8500=0.006 N. This means that in order to support the 50 N of load applied to the pull wire, the holding tension is reduced to only 0.006 N with 8 windings around the insert and the coefficient of friction of the stated materials. This beneficially allows for a significant reduction (e.g., 99.99% reduction in the above 50N example) in anchoring strength needed to withstand a high tension in the pull wire exiting the capstan on the distal end of the wire wraps. The above method may also be used to determine the number of windings based on the load and a coefficient of friction.

The insert component 401 may be formed of any suitable material. For instance, the insert component may be made of any suitable material such as stainless steel (e.g., SS316), metals, alloys, polymers, nylons or biocompatible material. The material of the insert may be selected to provide sufficient coefficient of friction at a contact interface between the insert and the pull wire. In some cases, a contact surface of the insert may be roughened to increase the fiction.

Experiments are conducted to show that the provided anchoring mechanism effectively prevent wire breakage. Additionally, tensile test to failure after cyclical loads show little difference with the pre-conditioned tensile strength of the wire. This beneficially allows the terminal of the pull wire be held to the insert in any suitable manner (e.g., tied knot, glued, mechanical fastener, etc.).

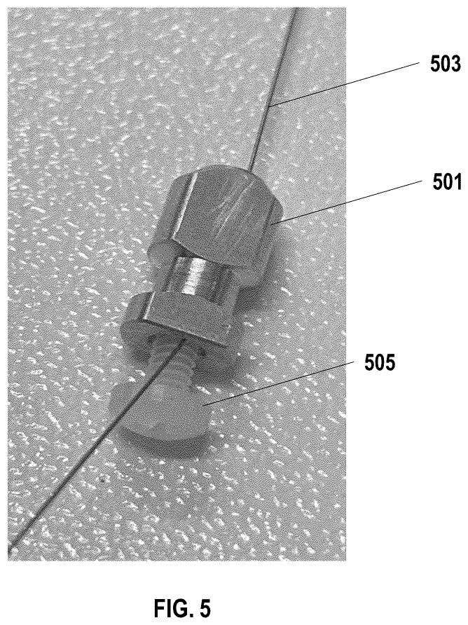

FIG. 5 shows an example of fixing the terminal end of a pull wire 503 to an insert component 501. As mentioned above, as the pre-conditioned tensile strength of the wire has little effect on the reduction of the holding tension, the provided anchoring mechanism allows for flexibility in fixing the wire terminal end to the insert. FIG. 5 shows an example of fixing the terminal end of the pull wire 503 to the insert component 501 using a screw 505. However, any other fastening mechanism such as crimps, adhesives, solder, welds, or knots may be utilized to hold the wire to the insert. The effect of reducing the holding tension at the terminal end may not be compromised by different selections of the fastening mechanism or coupling means. The flexibility of fixing the terminal end of the pull wire may also simplify the assembly process.

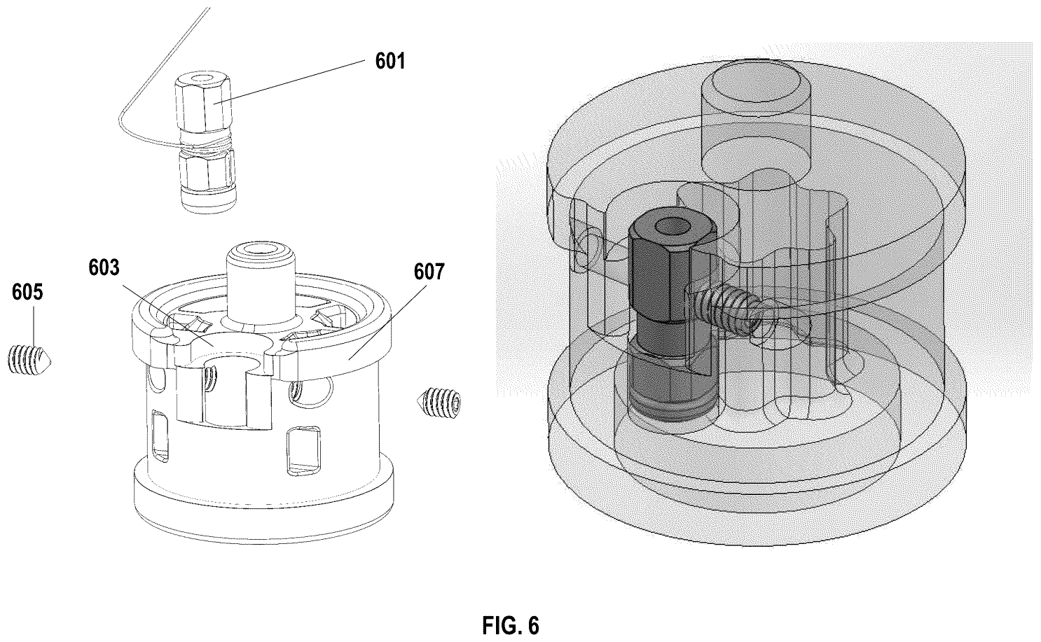

The insert component may be coupled to a capstan. In some cases, the insert component may be releasably coupled to a capstan. In some embodiments, the anchoring mechanism may comprise a feature to prevent a movement between the insert and the capstan. FIG. 6 shows an example of coupling the insert component 601 to a capstan 607.

In some embodiments, a feature for preventing a rotational movement between the insert component and the capstan may comprise a fastening mechanism to fix the insert component to the capstan. This effectively prevents further winding or unwinding of the pull wire. In some cases, the anchoring mechanism may be freely rotatable once it is positioned within a cutout 603 of the capstan and a fastening mechanism may be employed to prevent it from rotating. For example, a fastening mechanism 605 such as one or more side screws, dowel pin, shim, adhesive, and the like may be employed to fix the insert component 601 within the cutout 603 of the capstan 607. This may beneficially allow for the flexibility in adjusting the number of windings/wraps. For instance, pull wire winding may be adjusted while the insert component is positioned inside the capstan allowing for ease of assembly. For example, the insert component 601 may be freely rotating inside a recessive structure of the capstan 603 until a desired number of wraps around the insert is achieved, then the fastening element 605 (e.g., side screws) may be applied to fix the insert component 601 to the capstan 607. The cutout of the capstan 603 may have a dimension and/or shape configured to receive the insert component 601 and allow a free rotational movement of the insert component within the cutout where the shape of the cutout may not need to match the shape of the insert component.

In alternative embodiments, a feature for preventing a rotational movement between the insert and the capstan may be based on a shape of the insert and a shape of the cutout of the capstan. The anchoring mechanism may not be rotatable once it is placed within the cutout. The insert 601 and the cutout 603 of the capstan may be shaped and/or sized (e.g., non-circular shape with edges) such that the insert may not be able to rotate once it is assembled to the capstan. For example, the insert 601 may comprise a number of sides (e.g., six-sided head, five-sided head, four-sided head, three-sided head, etc.) and the cutout 603 of the capstan receiving the insert may have a matching shape and dimension thereby preventing a rotational movement. In such embodiments, the pull wire may be wrapped around the insert component prior to insertion into the capstan.

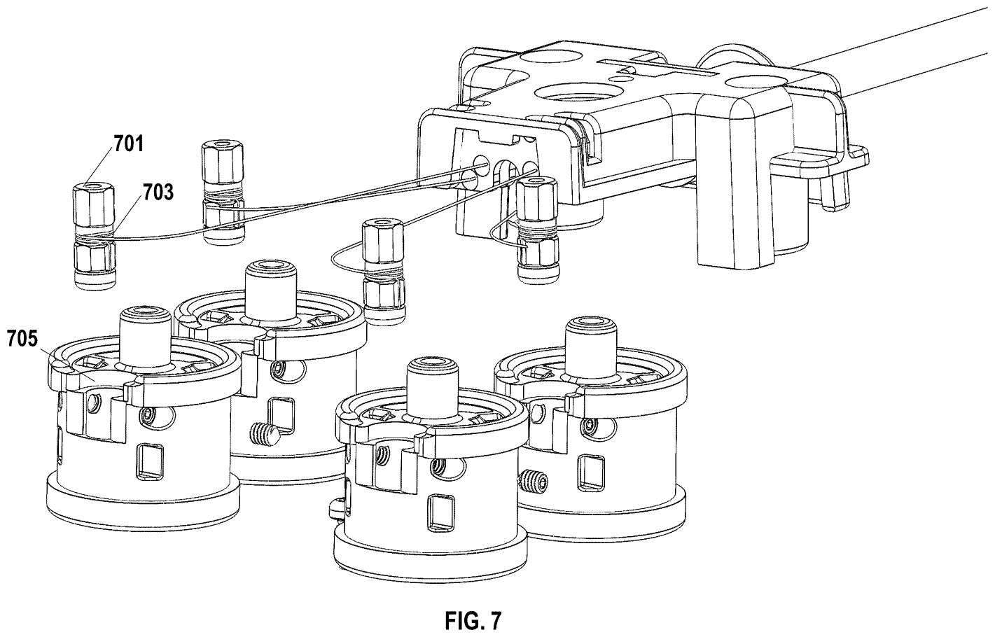

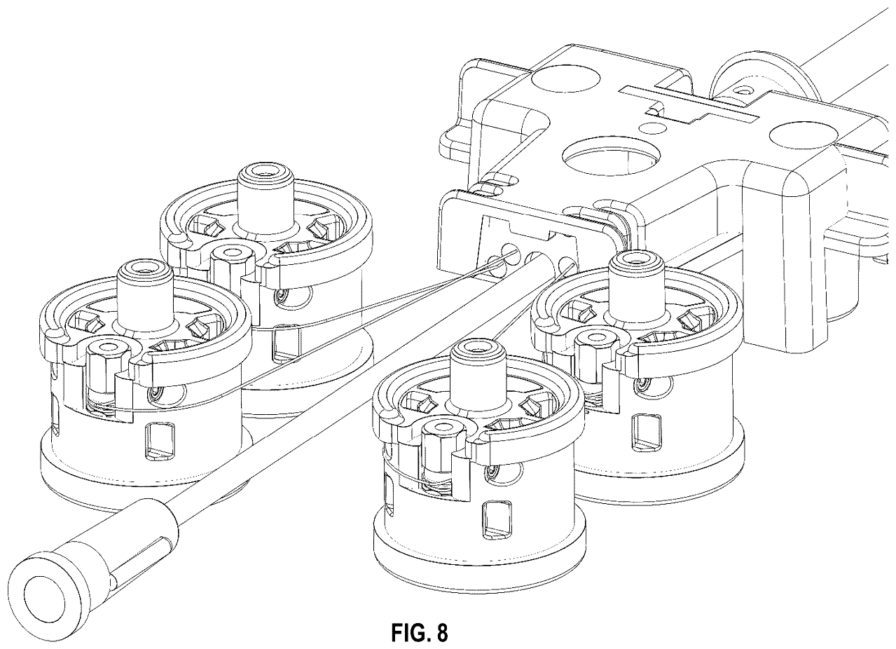

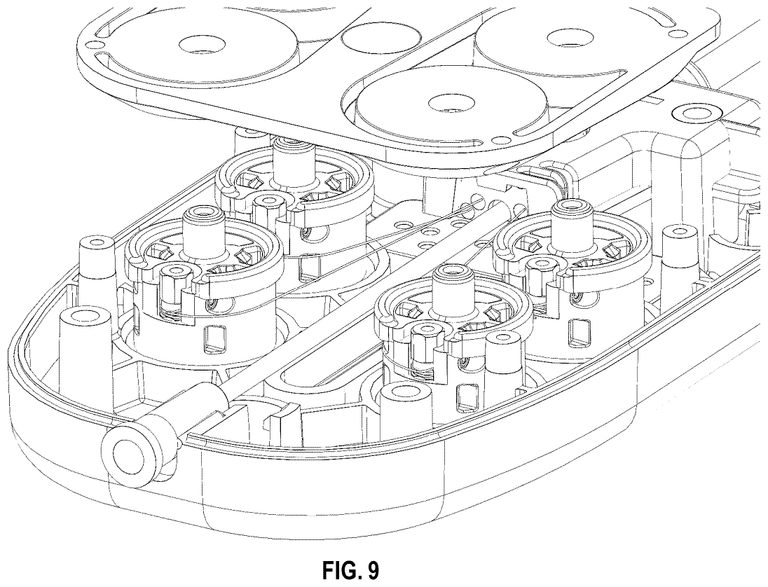

Once the proximal end of the pull wire is anchored to the capstan, the capstan may be assembled to the proximal portion of the endoscope. FIGS. 7-9 show examples of a proximal portion of the endoscope comprising a plurality of capstans and a plurality of corresponding anchoring mechanisms. In some cases, each capstan may correspond to an anchoring mechanism. Assembly of the proximal portion may comprise first assembling the anchoring mechanism to the capstan then assembling the capstan to the handle portion. For example, as shown in FIG. 7, a proximal end of a pull wire 703 may be coupled to an anchoring mechanism and wrapped around the insert component 701 with a predetermine number of windings, then the insert component with the pull wire may be placed inside a corresponding capstan 705. The number of windings may or may not be adjusted prior to fixing the insert component to the capstan such as using a screw.

### Low Cost and Single Use Robotic Endoscope (Bronchoscope)

The proximal design may improve the reliability of the device without introducing extra cost allowing for a low-cost single-use endoscope. In another aspect of the invention, a single-use robotic endoscope is provided. The robotic endoscope may be a bronchoscope and can be the same as the steerable catheter assembly as described elsewhere herein. Traditional endoscopes can be complex in design and are usually designed to be re-used after procedures, which require thorough cleaning, dis-infection, or sterilization after each procedure. The existing endoscopes are often designed with complex structures to ensure the endoscopes can endure the cleaning, dis-infection, and sterilization processes. The provided robotic bronchoscope can be a single-use endoscope that may beneficially reduce cross-contamination between patients and infections. In some cases, the robotic bronchoscope may be delivered to the medical practitioner in a pre-sterilized package and are intended to be disposed of after a single-use.

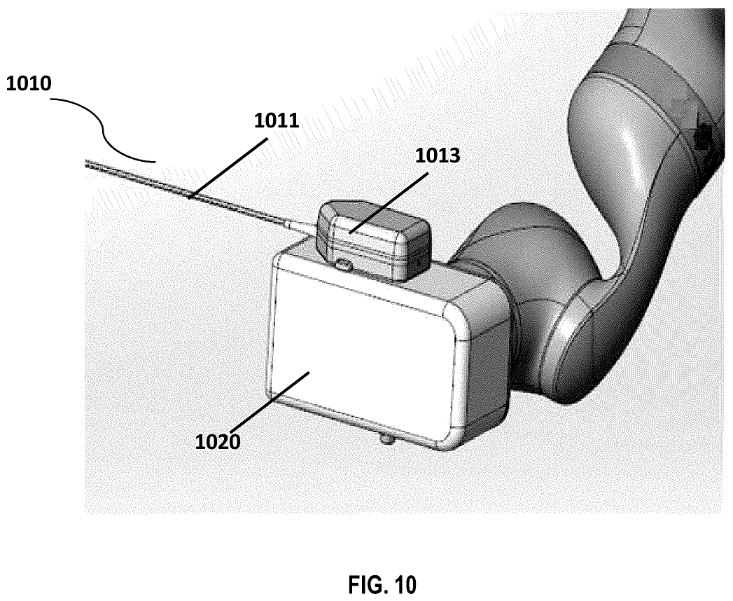

As shown in FIG. 10, a robotic bronchoscope 1020 may comprise a handle portion 1013 and a flexible elongate member 1011. In some embodiments, the flexible elongate member 1011 may comprise a shaft, steerable tip and a steerable section. The robotic bronchoscope 1020 can be the same as the steerable catheter assembly as described in FIG. 1. The robotic bronchoscope may be a single-use robotic endoscope. In some cases, only the catheter may be disposable. In some cases, at least a portion of the catheter may be disposable. In some cases, the entire robotic bronchoscope may be released from the instrument driving mechanism and can be disposed of. The bronchoscope may contain varying levels of stiffness along its shaft, as to improve functional operation.

The robotic bronchoscope can be releasably coupled to an instrument driving mechanism 1020. The instrument driving mechanism 1020 may be mounted to the arm of the robotic support system or to any actuated support system as described elsewhere herein. The instrument driving mechanism may provide mechanical and electrical interface to the robotic bronchoscope 1010. The mechanical interface may allow the robotic bronchoscope 1010 to be releasably coupled to the instrument driving mechanism. For instance, the handle portion of the robotic bronchoscope can be attached to the instrument driving mechanism via quick install/release means, such as magnets and spring-loaded levels. In some cases, the robotic bronchoscope may be coupled or released from the instrument driving mechanism manually without using a tool.

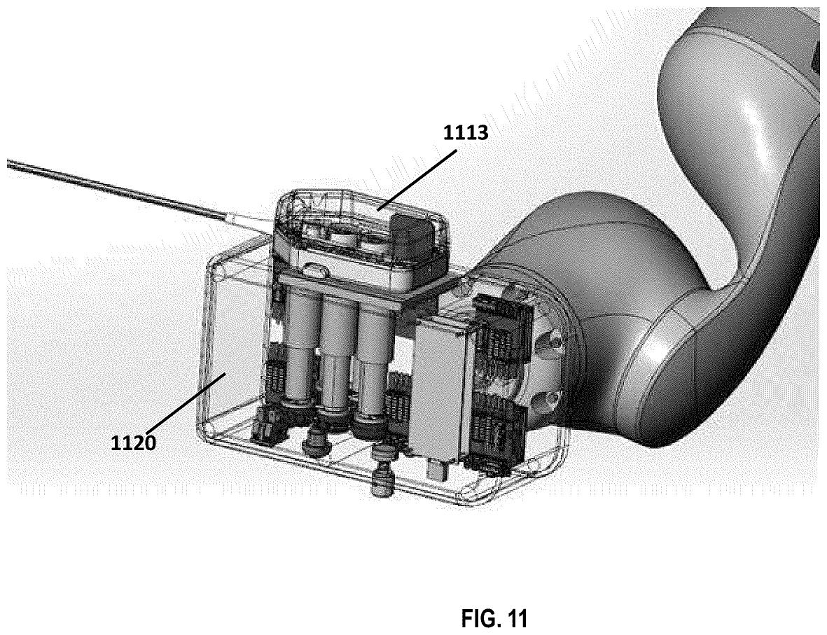

FIG. 11 shows an example of an instrument driving mechanism 1120 providing mechanical interface to the handle portion 1113 of the robotic bronchoscope. As shown in the example, the instrument driving mechanism 1120 may comprise a set of motors that are actuated to rotationally drive a set of pull wires of the flexible endoscope or catheter. The handle portion 1113 of the catheter assembly may be mounted onto the instrument drive mechanism so that its pulley assemblies or capstans are driven by the set of motors. The number of pulleys may vary based on the pull wire configurations. In some cases, one, two, three, four, or more pull wires may be utilized for articulating the flexible endoscope or catheter.

The handle portion may be designed allowing the robotic bronchoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive, adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

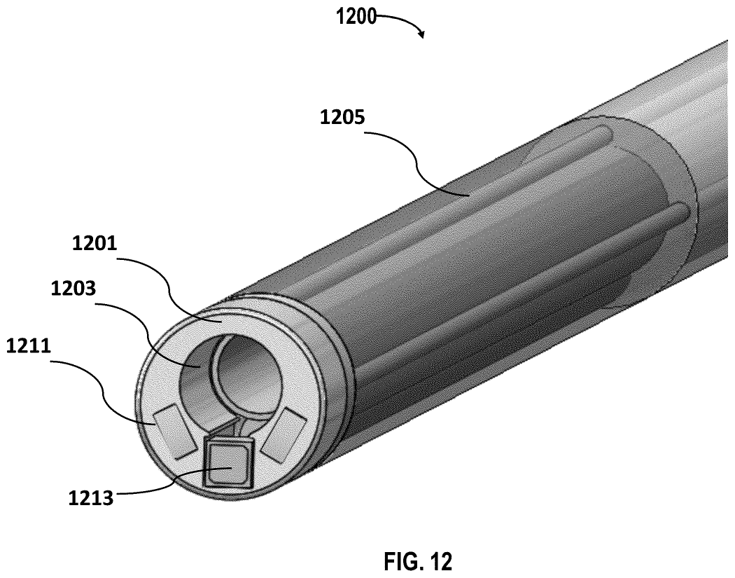

FIG. 12 shows an example of a distal tip 1200 of an endoscope. In some cases, the distal portion or tip of the catheter 1200 may be substantially flexible such that it can be steered into one or more directions (e.g., pitch, yaw). The catheter may comprise a tip portion, bending section, and insertion shaft. In some embodiments, the catheter may have variable bending stiffness along the longitudinal axis direction. For instance, the catheter may comprise multiple segments having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some cases, a proximal end of the catheter needs not be bent to a high degree thus the proximal portion of the catheter may be reinforced with additional mechanical structure (e.g., additional layers of materials) to achieve a greater bending stiffness. Such design may provide support and stability to the catheter. In some cases, the variable bending stiffness may be achieved by using different materials during extrusion of the catheter. This may advantageously allow for different stiffness levels along the shaft of the catheter in an extrusion manufacturing process without additional fastening or assembling of different materials.

The distal portion of the catheter may be steered by one or more pull wires 1205. The distal portion of the catheter may be made of any suitable material such as co-polymers, polymers, metals or alloys such that it can be bent by the pull wires. In some embodiments, the proximal end or terminal end of one or more pull wires 1205 may be coupled to a driving mechanism (e.g., gears, pulleys, capstan etc.) via the anchoring mechanism as described above.

The pull wire 1205 may be a metallic wire, cable or thread, or it may be a polymeric wire, cable or thread. The pull wire 1205 can also be made of natural or organic materials or fibers. The pull wire 1205 can be any type of suitable wire, cable or thread capable of supporting various kinds of loads without deformation, significant deformation, or breakage. The distal end or portion of one or more pull wires 1205 may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

As described above, the pull wires may be made of any suitable material such as stainless steel, metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals. Pull wires may run through the lumen of one or more load transmission tubes and/or hypotubes as described elsewhere herein.

The catheter may have a dimension so that one or more electronic components can be integrated to the catheter. For example, the outer diameter of the distal tip may be around 4 to 4.4 millimeters (mm), and the diameter of the working channel may be around 2 mm such that one or more electronic components can be embedded into the wall of the catheter. However, it should be noted that based on different applications, the outer diameter can be in any range smaller than 4 mm or greater than 4.4 mm, and the diameter of the working channel can be in any range according to the tool dimensional or specific application.

The one or more electronic components may comprise an imaging device, illumination device or sensors. In some embodiments, the imaging device may be a video camera 1213. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 1211 positioned at the distal tip. The light source may be a light-emitting diode (LED), an organic LED (OLED), a quantum dot, or any other suitable light source. In some cases, the light source may be miniaturized LED for a compact design or Dual Tone Flash LED Lighting.

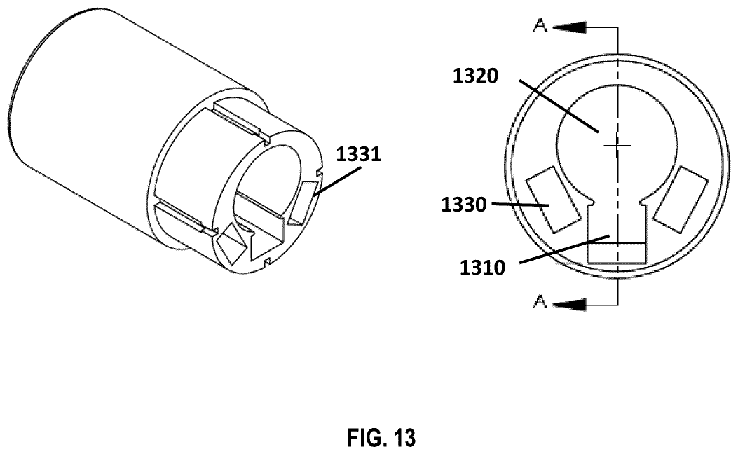

The imaging device and the illumination device may be integrated to the catheter. For example, the distal portion of the catheter may comprise suitable structures matching at least a dimension of the imaging device and the illumination device. The imaging device and the illumination device may be embedded into the catheter. FIG. 13 shows an example distal portion of the catheter with integrated imaging device and the illumination device. A camera may be located at the distal portion. The distal tip may have a structure to receive the camera, illumination device and/or the location sensor. For example, the camera may be embedded into a cavity 1310 at the distal tip of the catheter. The cavity 1310 may be integrally formed with the distal portion of the cavity and may have a dimension matching a length/width of the camera such that the camera may not move relative to the catheter. The camera may be adjacent to the working channel 1320 of the catheter to provide near field view of the tissue or the organs. In some cases, the attitude or orientation of the imaging device may be controlled by controlling a rotational movement (e.g., roll) of the catheter.

The power to the camera may be provided by a wired cable. In some cases, the cable wire may be in a wire bundle providing power to the camera as well as illumination elements or other circuitry at the distal tip of the catheter. The camera and/or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure 1330 having a dimension matching a dimension of the miniaturized LED light source. As shown in the illustrated example, two cavities 1330 may be integrally formed with the catheter to receive two LED light sources. For instance, the outer diameter of the distal tip may be around 4 to 4.4 millimeters (mm) and diameter of the working channel of the catheter may be around 2 mm such that two LED light sources may be embedded at the distal end. The outer diameter can be in any range smaller than 4 mm or greater than 4.4 mm, and the diameter of the working channel can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiment, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover 1331 may be placed at the forwarding end face of the distal tip providing precise positioning of the LEDs as well as sufficient room for the glue. The cover 1331 may be composed of transparent material matching the refractive index of the glue so that the illumination light may not be obstructed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An articulating flexible endoscope comprising:
a proximal portion comprising a driving mechanism for applying a force to a pull wire;
the pull wire for articulating a bending section of the articulating flexible endoscope upon the force; and
an anchoring mechanism coupled to the driving mechanism, wherein a proximal end of the pull wire is configured to wind around the anchoring component with a pre-determined number of windings thereby reducing a holding tension at the proximal end of the pull wire.

2. The articulating flexible endoscope of claim 1, wherein the driving mechanism comprises a capstan.

3. The articulating flexible endoscope of claim 2, wherein the capstan comprises a cutout to receive the anchoring mechanism.

4. The articulating flexible endoscope of claim 3, wherein the anchoring mechanism is freely rotatable within the cutout.

5. The articulating flexible endoscope of claim 4, wherein the anchoring mechanism is rotated to adjust the number of windings.

6. The articulating flexible endoscope of claim 1, wherein the anchoring mechanism is coupled to the driving mechanism via a screw.

7. The articulating flexible endoscope of claim 3, wherein the anchoring mechanism and the cutout have a non-circular shape such that the anchoring mechanism is not rotatable within the cutout.

8. The articulating flexible endoscope of claim 1, wherein the pre-determined number of windings are determined based at least in part on a load requirement and a coefficient of friction between the pull wire and the anchoring mechanism.

9. The articulating flexible endoscope of claim 1, further comprising a distal tip portion embedded with an imaging device, a position sensor and an illumination device.

10. The articulating flexible endoscope of claim 9, wherein the entire articulating flexible endoscope is disposable or single-use.

11. A method for coupling a pull wire to an articulating flexible endoscope comprising:
(a) providing an anchoring mechanism;
(a) wrapping a proximal end of the pull wire to the anchoring mechanism with a pre-determined number of windings thereby reducing a holding tension at the proximal end of the pull wire; and
(b) assembling the anchoring mechanism to a driving mechanism located at a proximal portion of the articulating flexible endoscope.

12. The method of claim 11, wherein the driving mechanism is configured for applying a force to a pull wire.

13. The method of claim 12, wherein a bending section of the articulating flexible endoscope is articulated by the pull wire upon the force.

14. The method of claim 11, wherein the driving mechanism comprises a capstan.

15. The method of claim 14, wherein the capstan comprises a cutout to receive the anchoring mechanism.

16. The method of claim 15, wherein the anchoring mechanism is freely rotatable within the cutout.

17. The method of claim 16, further comprising adjusting the number of windings by rotating the anchoring mechanism.

18. The method of claim 11, further comprising determining the pre-determined number of windings based at least in part on a load requirement and a coefficient of friction between the pull wire and the anchoring mechanism.

19. The method of claim 11, wherein the articulating flexible endoscope comprises a distal tip portion embedded with an imaging device, a position sensor and an illumination device.

20. The method of claim 19, wherein the entire articulating flexible endoscope is disposable or single-use.
